(19) United States
(12) Patent Application Publication
(10) Pub. No.: US 2020/0179339 A1
(43) Pub. Date: Jun. 11, 2020

(54) PHARMACEUTICALS COMPOSITIONS FOR TREATMENT OF ATOPIC DERMATITIS

(71) Applicant: CADILA HEALTHCARE LIMITED, Ahmedabad (IN)

(72) Inventors: Mukul R. JAIN, Ahmedabad (IN); Abhijit CHATTERJEE, Ahmedabad (IN); Kaushik BANERJEE, Ahmedabad (IN); Jaideep SINGH, Ahmedabad (IN); Sunita SINGH, Ahmedabad (IN); Jitendrakumar PATEL, Ahmedabad (IN)

(21) Appl. No.: 16/704,447

(22) Filed: Dec. 5, 2019

(30) Foreign Application Priority Data
Dec. 7, 2018 (IN) 201821046429

(51) Int. Cl. A61K 31/4015; A61K 31/4025; A61K 31/196; A61K 9/06; A61K 47/38; A61K 47/10; A61K 47/18; A61K 47/22; A61K 47/12; A61P 17/00

(52) U.S. Cl. CPC A61K 31/4015; A61K 31/4025; A61K 31/196; A61K 9/06; A61K 47/38; A61K 9/0014; A61K 47/186; A61K 47/22; A61K 47/12; A61P 17/00; A61K 47/10

(57) ABSTRACT

The present invention provides a combination of PDE4 inhibitors or pharmaceutically acceptable salts thereof along with at least one secondary therapeutic agent. The present invention also provides process for the preparation of topical pharmaceutical composition of said combination with one or more pharmaceutically acceptable excipients. The present invention further relates to use of topical pharmaceutical composition for the treatment of atopic dermatitis.

### FIELD OF THE INVENTION

The present invention provides a combination of PDE4 inhibitors or pharmaceutically acceptable salts thereof along with at least one secondary therapeutic agent. The present invention also provides process for the preparation of topical pharmaceutical composition of said combination with one or more pharmaceutically acceptable excipients. The present invention further relates to use of topical pharmaceutical composition for the treatment of atopic dermatitis.

### BACKGROUND OF THE INVENTION

Atopic dermatitis (AD) is a chronic disease of the skin based on skin barrier dysfunction, which together with various environmental factors and multiple changes of the immune system leads to eczematous and itchy lesions at the flexural folds and other typical distributions (Schultz et al, J Am Acad Dermatol.; 34 (1996): 760-764). Acute flare-ups and exacerbations as well as chronic eczematous skin lesions on dry skin accompanied by intensive pruritus are other characteristics of this disease.

Phosphodiesterase (PDE) 4 inhibitors are a class of low molecular weight compounds that exhibit anti-inflammatory effects (Spina D. et al, British Journal of Pharmacology 155.3 (2008): 308-315). Clinically, PDE4 inhibitors have been employed for the treatment of various chronic inflammatory diseases, including psoriasis, psoriatic arthritis, atopic dermatitis, and inflammatory bowel disease (Schafer et al, Biochemical Pharmacology 83.12 (2012); 1583-1590).

Apremilast is an oral PDE4 inhibitor that works intracellularly to regulate production of pro- and anti-inflammatory mediators implicated in the pathogenesis of psoriasis and psoriatic arthritis. Apremilast is clinically used for the treatment of adult patients with active psoriatic arthritis and for patients with moderate to severe plaque psoriasis (Young et al, Journal of the American Association of Nurse Practitioners 28.12 (2016): 683-695).

Apremilast is presently available in market under the tread name of OTEZLA® by the Celgene group. OTEZLA® is marketed orally in 10 mg, 20 mg and 30 mg strength. Apremilast is generally well tolerated orally, with the most common adverse events being weight loss, diarrhea, nausea and emesis in the first year of treatment and nasopharyngitis and upper respiratory tract infection with continued treatment (Keating et al, Drugs 77.4 (2017): 459-472).

Common side effects related to oral Apremilast are diarrhea, headache, nausea, vomiting, weight loss and depression. These side effects are dose-limiting and thus the therapeutic potential of Apremilast may not be fully realized. (Langley et al, Journal of Cutaneous Medicine and Surgery, 22(4), (2017). 415-421 and Rosso et al J Clin Aesthet Dermatol.;9(9): (2016): 43-48). To overcome these obstacles, the topical route of administration of Apremilast with potent and long-lasting local effect and minimal systemic exposure has been proposed.

WIPO patent application no. WO 2014151180 discloses methods of treating or preventing psoriatic arthritis using apremilast in oral dosage form.

WIPO patent application no. WO 2018138737 discloses topical pharmaceutical compositions comprising Apremilast and relates to methods for preparing such compositions and methods of using them in management of skin diseases or disorders.

WIPO patent application no. WO 2017216738 discloses topical formulation of apremilast with dimethyl isosorbide.

Non-steroidal anti-inflammatory drugs (NSAID) are the most widely used drugs in the world for the variety of inflammatory condition. As inflammation is a common symptom of many of skin diseases, NSAIDs play an important therapeutic role in dermatology (Friedman, E. S et al 2002)

Diclofenac sodium, 2-[(2,6-dichlorophenyl) amino] benzene acetic acid, is a non-steroidal anti-inflammatory drug (NSAID) with an approximate relative COX-1/COX-2 specificity ratio of one. NSAIDs inhibit the cyclo-oxygenase enzymes (COX) and decrease peripheral and central prostaglandin production.

The current formulations of oral and topical PDE4 inhibitors has lot of side effects, longer duration of treatment and these therapies are not able to cure completely. The topical combination of the present invention PDE4 inhibitor and NSAIDs has significant advantages over other therapies known for atopic dermatitis and may show significant improvement in atopic dermatitis.

### EMBODIMENTS OF THE INVENTION

In an embodiment, the present invention provides a combination comprising PDE4 inhibitor and at least one secondary therapeutic agent.

In another embodiment, the present invention provides Apremilast as a PDE4 inhibitor.

In another embodiment, the present invention provides a pharmaceutical composition comprising compound of PDE4 inhibitor and at least one or more secondary therapeutic agent which is selected from a non-steroidal anti-inflammatory drug (NSAID).

In another further embodiment, the present invention provides a topical pharmaceutical composition comprising Apremilast and an anti-inflammatory agent as a second therapeutic agent.

In preferred embodiment, the present invention provides Diclofenac sodium as the anti-inflammatory agent (NSAID).

In a preferred embodiment, the present invention provides Salsalate as the anti-inflammatory agent.

In another preferred embodiment, the present invention provides a topical pharmaceutical composition comprising Apremilast, Diclofenac sodium and other necessary pharmaceutically acceptable excipients.

In another preferred embodiment, the present invention provides a topical pharmaceutical composition comprising Apremilast, Salsalate and other necessary pharmaceutically acceptable excipients.

In another preferred embodiment, the present invention provides a topical pharmaceutical composition comprising Apremilast, diclofenac sodium, Salsalate and other necessary pharmaceutically acceptable excipients.

In still preferred embodiment, the present invention provides a topical, pharmaceutical composition comprising Apremilast, Diclofenac sodium and other necessary pharmaceutically acceptable excipients for prevention and treatment of atopic dermatitis.

In another preferred embodiment, the present invention provides a topical pharmaceutical composition comprising Apremilast, diclofenac sodium, Salsalate and other necessary pharmaceutically acceptable excipients for prevention and treatment of atopic dermatitis.

### DESCRIPTION OF FIGURES

FIG. 1: The effect of Apremilast (3%) alone, Diclofenac (1.5%) alone and their combination on ear thickness in oxazolone induced atopic dermatitis in mice

FIG. 2: The effect of Apremilast (1%) alone, Diclofenac (1%) alone and their combination on ear thickness in oxazolone induced atopic dermatitis in mice

### DETAILED DESCRIPTION OF THE INVENTION

The term ‘treating’ or ‘treatment’ or condition as used herein means: preventing or delaying the appearance of clinical symtomps of the state, disorder or condition developing in a mammal.

The term “preventing” refers to barring a subject from acquiring a disorder or disease in the first place.

The term ‘pharmaceutically acceptable’ use embraces both human and veterinary use.

The term ‘topical composition’ or ‘topical formulation’ means a composition in which the drug may be placed for direct application to a skin surface and from which an effective amount of the drug is released. Such formulation may include creams, ointments, gels, lotions or any other dosage form suitable for topical application and the like. In some aspects, such formulation may be applied to the skin in an unoccluded form with/without additional backing, structures or devices.

The term ‘skin’ or ‘skin surface’ is meant to include the outer skin of a subject comprising one or more of epidermal layers to which a drug composition may be administered.

The topical formulation of the present invention include those suitable for topical, transdermal, rectal and buccal (e,g, sub-lingual) administration etc.; preferably the formulation of the present invention are administered topically and are provided in the form of semisolid dosage forms. Suitable dosage forms include hydrous or anhydrous semisolids such as creams, ointments, gels, lotions or any other dosage forms suitable for topical application.

The present invention relates to topical pharmaceutical composition comprising PDE4 inhibitor in combination with at least one secondary therapeutic agent for prevention or treatment of atopic dermatitis.

As provided in an embodiment, the PDE4 inhibitor is (+)-2-[1-(3-ethoxy-4-methoxyphenyl)-2-methanesulfonylethyl]-4-acetylaminoisoindolin-1,3-dione, generally known as Apremilast, which has the following chemical structure

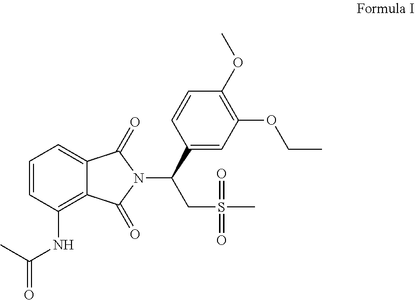 [SMILES: CCOc1cc([C@@H](CS(C)(=O)=O)N2C(=O)c3cccc(NC(C)=O)c3C2=O)ccc1OC]

or pharmaceutically acceptable salt or solvate or hydrate thereof.

In an embodiment, the second therapeutic agent is selected from suitable non-steroidal anti-inflammatory agents.

In a preferred embodiment, suitable non-steroidal anti-inflammatory agents are selected from diclofenac, naproxen, ibuprofen, ketoprofen, piroxicam, meloxicam, salsalate, aceclofenac or suitable combinations thereof.

According to one of the preferred embodiment, the anti-inflammatory agent is sodium salt of [2-(2,6-Dichloroanilino)phenyl]acetic acid which is known as Diclofenac sodium.

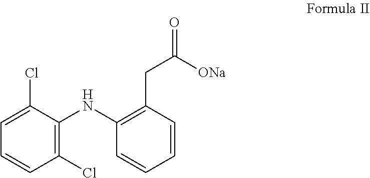

In another preferred embodiment, the anti-inflammatory agent is 2-(2-Hydroxybenzoyl)oxybenz oic acid or known as Salsalate.

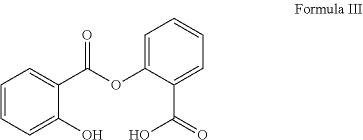 [SMILES: O=C(Oc1ccccc1C(=O)O)c1ccccc1O]

or its pharmaceutically acceptable salt.

In another preferred embodiment, the present invention provides a topical, pharmaceutical composition comprising Apremilast, Diclofenac sodium and other necessary pharmaceutically acceptable excipients and preparation thereof.

In another preferred embodiment, the present invention provides a topical, pharmaceutical composition comprising Apremilast, Salsalate and other necessary pharmaceutically acceptable excipients and preparation thereof.

In another preferred embodiment, the present invention provides a topical pharmaceutical composition comprising Apremilast, Diclofenac sodium, Salsalate and other necessary pharmaceutically acceptable excipients and preparation thereof.

In a preferred embodiment, the present invention discloses a topical formulation comprising Apremilast and Diclofenac sodium present at a concentration of (0.01 to 10%)w/w and (0.01 to 10%)w/w respectively.

In a preferred embodiment, the present invention discloses a topical formulation comprising Apremilast and Salsalate present at a concentration of (0.01 to 10%)w/w and (0.01 to 10%)w/w respectively.

In a preferred embodiment, the present invention discloses a topical formulation comprising Apremilast present at a concentration of (0.01 to 10%)w/w, Diclofenac sodium present at a concentration of (0.01 to 10%)w/w and Salsalate present at a concentration of (0.01 to 10%)w/w, respectively.

In an embodiment topical formulation of the present invention includes gel, ointment, cream, lotion, solution and foams and process for the preparation thereof.

In an embodiment suitable pharmaceutically acceptable excipients are selected from thickening agents, neutralizing agents, penetration enhancers, vehicle, preservatives, fillers, propellant or surfactant.

In a preferred embodiment, thickening agents are selected from carbomer polymer, carbomer derivative, cellulose derivatives, anionic polymers, polyvinyl alcohol, water soluble polysaccharide, pectin, arabic gum, karaya gum, tragacanth gum, glycols or Polysorbate 40.

In a preferred embodiment, penetration enhancers are selected from isopropyl palmitate, myristic acid, oleic acid, oleyl alcohol, Pyrrolidone, isopropyl myristate, some aliphatic alcohols selected from ethanol, monohydric/polyhydric alcohols.

In a preferred embodiment, neutralizing agents are selected from polyfunctional amine such as triethanolamine, poly(ethyleneimine), diisopropanolamine, triisopropanolamine, arginine, aminomethyl propanol, tetrahydroxypropyl ethyl enediamine or tromethamine

In a preferred embodiment, vehicle is selected from the group of pharmaceutically acceptable glycols (for example, propyleneglycol and hexylene glycol, diethylene glycol monoethyl ether) alcohols, polysorbate 40 (a polyhydroxy organic compound), dimethyl sulphoxide, 1, 2-, diols, and mixtures thereof.

In a preferred embodiment, preservative is selected from acids, esters, alcohols, phenols selected from butylated hydroxy anisol, butylated hydroxy toluene, and quaternary ammonium compounds.

In a preferred embodiment, propellant or surfactant is selected from the group comprising of hydrocarbons selected from propane and butane, a chlorofluorocarbon dimethyl ether, hydroflourocarbon and suitable mixtures thereof.

The other suitable pharmaceutical carrier are selected from conventional excipients such as binding agents, fillers, lubricants, glidants, disintegrants and wetting agents.

In an embodiment, the topical composition of the present invention are suitable for the prevention or treatment of Atopic dermatitis.

The invention is further exemplified by the following non-limiting examples, which are illustrative representing the preferred modes of carrying out the invention. The invention's scope is not limited to these specific embodiments only but should be read in conjunction with what is disclosed anywhere else in the specification together with those information and knowledge which are within the general understanding of a person skilled in the art.

### Biological Studies:

### Protocol for Pharmacological Studies Data:

### Study Design and Treatment

Study-1: Treatment with Topical Combination of Diclofenac (1.5%) and Apremilast (3%) in Ear Thickness in Oxazolone Induced Atopic Dermatitis in Mice

The 7-8 week old female balb/c mice used for the study were divided into four groups of six mice each. Animal shaved on their abdomen region. On day-1, Oxazolone (Sigma chemical Co. USA) was dissolved in ethanol and applied at a dose volume is 500 ug/100 μl/mouse on shaved abdomen side as sensitized dose. On day 5, basal ear thickness was measured and oxazolone (dissolved in acetone, (100 μg/20 μl)) applied on left side ear at a dose volume of 20 μl/mouse on day 5 and day 6. Post Two hours of oxazolone application, treatment drugs applied on left ear respectively. Treatment was applied on day 5 and day 6 and ear thickness was measured on day 5 (basal), day 6 and day 7.

All animals were assessed for the ear thickness measurement. Animals were sacrificed on day 7, Ear weight were taken and ear were collected for histology and gene expression.

TABLE 1
Study

| Sr. | | Atopic dermatitis | |
|---|---|---|---|
| No. | Groups | inducer | Treatment |
| 1 | Normal control | Oxazolone | Treatment applied |
| 2 | Vehicle control (VC) | application | topically from day |
| 3 | VC + Apremilast (3%) | | 5 to day 6. |
| 4 | VC + Diclofenac (1.5%) | | |
| 5 | VC + Apremilast (3%) + | | |
| | Diclofenac (1.5%) | | |

Table-2 below shows effect of Diclofenac (1.5%) and Apremilast (3%) and their combination in ear thickness in oxazolone induced atopic dermatitis in mice.

TABLE 2

| Groups | Change in ear thickness | | | % change | | |
|---|---|---|---|---|---|---|
| Vehicle Control | 0.3 | ± | 0.02 | | | |
| Apremilast (3%) | 0.18 | ± | 0.01 | 38.6 | ± | 3.55 |
| Diclofenac (1.5%) | 0.11 | ± | 0.01 | 63.8 | ± | 3.52 |
| Apremilast (3%) + | 0.06 | ± | 0.01 | 80 | ± | 3.56 |
| Diclofenac (1.5%) | | | | | | |

### Results:

Topical combination of Apremilast (3%) and Diclofenac (1.5%) has shown improvement in ear thickness in oxazolone induced atopic dermatitis in mice.

Post challenged and sensitization by topical application of oxazolone, there is a significant increase in ear thickness in all animals.

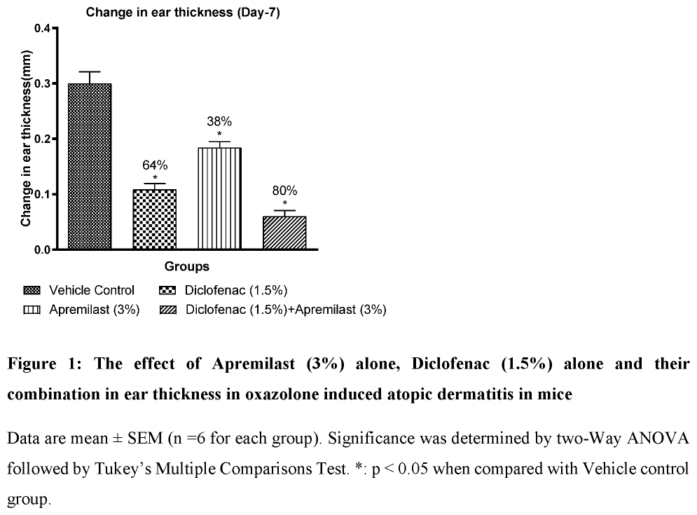

The percentage inhibition in ear thickness (atopic dermatitis) was significantly higher in combination group (80%) when compared with Apremilast (38%) and Diclofenac (64%) alone group indicating improvement in atopic dermatitis (Table 2 and FIG. 1). Application of vehicle alone did not significantly affect oxazolone induced atopic dermatitis in mice.

### Conclusion:

Topical combination of Apremilast (3%) with Diclofenac (1.5%) has shown improvement in atopic dermatitis condition. The combination of Apremilast with Diclofenac has shown improvement in ear thickness when compared with Apremilast and Diclofenac alone.

Study-2: Treatment with Topical Combination of Diclofenac (1%) and Apremilast (1%) in Ear Thickness in Oxazolone Induced Atopic Dermatitis in Mice

The 7-8 week old female balb/c mice used for the study were divided into five groups of six mice each. Animal shaved on their abdomen region. On day-1, Oxazolone (Sigma chemical Co. USA) was dissolved in ethanol and applied at a dose volume is 500 ug/100 μl/mouse on shaved abdomen side as sensitized dose. On day 5, basal ear thickness was measured and oxazolone (dissolved in acetone, (100 μg/20 μl)) applied on left side ear at a dose volume of 20 μ/mouse on day 5 and day 6. Post Two hours of oxazolone application, treatment drugs applied on left ear respectively. Treatment was applied on day 5 and day 6 and ear thickness was measured on day 5 (basal), day 6 and day 7.

All animals were assessed for the ear thickness measurement. Animals were sacrificed on day 7, Ear weight were taken.

TABLE 3
Study

| Sr. | | Atopic dermatitis | |
|---|---|---|---|
| No. | Groups | inducer | Treatment |
| 1 | Normal control | Oxazolone | Treatment applied |
| 2 | Vehicle control (VC) | application | topically from day |
| 3 | VC + Apremilast (1%) | | 5 to day 6. |
| 4 | VC + Diclofenac (1%) | | |
| 5 | VC + Apremilast(1%) + | | |
| | Diclofenac (1%) | | |

Table-4 below shows effect of Diclofenac (1%) and Apremilast (1%) and their combination in ear thickness in oxazolone induced atopic dermatitis in mice.

TABLE 4

| Groups | Change in ear thickness | | | % change | | |
|---|---|---|---|---|---|---|
| Vehicle Control | 0.44 | ± | 0.02 | | | |
| Apremilast (1%) | 0.24 | ± | 0.03 | 45.1% | ± | 6.58 |
| Diclofenac (1%) | 0.28 | ± | 0.01 | 36.8% | ± | 3.14 |
| Apremilast (1%) + | 0.10 | ± | 0.01 | 77.4% | ± | 3.19 |
| Diclofenac (1%) | | | | | | |

### Results:

Topical combination of Diclofenac (1%) and Apremilast (1%) has shown improvement in ear thickness in oxazolone induced atopic dermatitis in mice.

Post challenged and sensitization by topical application of oxazolone, there is a significant increase in ear thickness in all animals.

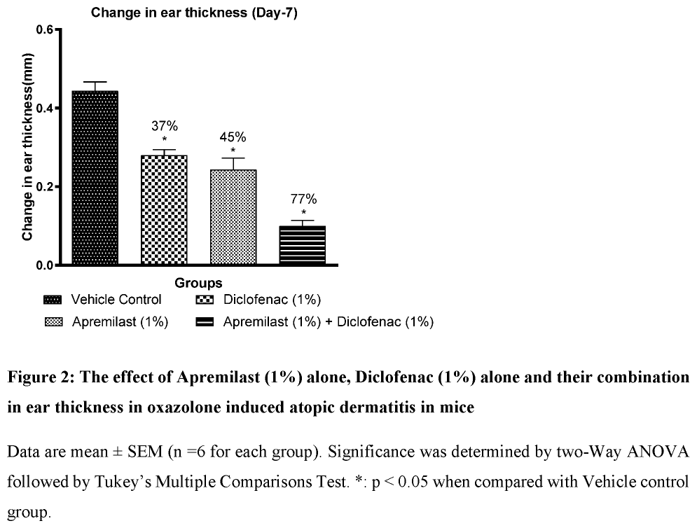

The percentage inhibition in ear thickness (atopic dermatitis) was significantly higher in combination group (77%) when compared with Apremilast (45%) and Diclofenac (37%) alone group indicating improvement in atopic dermatitis (Table-4 and FIG. 2). Application of vehicle alone did not significantly affect oxazolone induced atopic dermatitis in mice.

### Conclusion

Based on results as mentioned above, the combination of Diclofenac and Apremilast is showing significant reduction in ear thickness when compared with Diclofenac and Apremilast individually. Also, the combination (1% Diclofenac with 1% Apremilast) is showing similar effect (77%) compared with high doses of combination (80%). The combination can be served as a valuable tool for treatment for atopic dermatitis as it has advantage over current therapy such as steroids. The above combination will be a novel therapy, which is showing good efficacy and has advantage over current therapy such as less side effects, duration of treatment and cost effective.

1. A topical pharmaceutical composition comprising (i) a compound of formula (I) 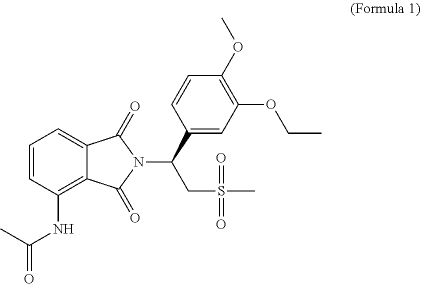 [SMILES: CCOc1cc([C@@H](CS(C)(=O)=O)N2C(=O)c3cccc(NC(C)=O)c3C2=O)ccc1OC]
(ii) at least one additional therapeutic agent selected from non-steroidal anti-inflammatory agent;
(iii) optionally one or more pharmaceutically acceptable excipient.

2. The pharmaceutical composition as claimed in claim 1, wherein non-steroidal anti-inflammatory agent is selected from diclofenac and its sodium salt, naproxen, ibuprofen, ketoprofen, piroxicam, meloxicam, salsalate, aceclofenac or suitable combinations thereof.

3. The pharmaceutical composition as claimed in claim 2, wherein non-steroidal anti-inflammatory agent is diclofenac sodium.

4. The topical pharmaceutical composition claim 1, wherein the compound of formula (I) is present in an amount of from 0.01 to 10% w/w and diclofenac sodium is present in an amount of from 0.01 to 10% w/w.

5. The pharmaceutical composition as claimed in claim 1, wherein the compound of formula (I) is present in an amount of from 0.5 to 10% w/w and the diclofenac sodium is present in an amount of from 0.5 to 10% w/w.

6. The pharmaceutical composition as claimed in claim 1, when applied topically, can be in the form of a gel, ointment, creams, lotion, solution and foams.

7. The pharmaceutical composition as claimed in claim 1 for use as an active therapeutic substance.

8. The pharmaceutical composition as claimed in claim 7 for use in the treatment of atopic dermatitis.

9. The topical pharmaceutical composition as claimed in claim 7 for use in the prevention or delay of progression of atopic dermatitis.

10. The topical pharmaceutical composition as claimed in claim 1, wherein pharmaceutical acceptable excipients is selected from thickening agents, neutralizing agent, penetration enhancers, vehicle, preservatives, propellant or surfactant.

11. The topical pharmaceutical composition as claimed in claim 10 wherein thickening agents are selected from carbomer polymer, carbomer derivative, cellulose derivatives, anionic polymers, polyvinyl alcohol, water soluble polysaccharide, pectin, arabic gum, karaya gum, tragacanth gum, glycols or Polysorbate 40.

12. The topical composition as claimed in claim 10, wherein neutralizing agents are polyfunctional amine selected from triethanolamine, poly(ethyleneimine), diisopropanolamine, triisopropanolamine, arginine, aminomethyl propanol, tetrahydroxypropyl ethylenediamine or tromethamine.

13. The topical composition as claimed in claimed 10, wherein penetration enhancers are selected from isopropyl palmitate, myristic acid, oleic acid, oleyl alcohol, Pyrrolidone, isopropyl myristate, some aliphatic alcohols selected from ethanol, or monohydric/polyhydric alcohols.

14. The topical composition as claimed in claim 10, wherein vehicle is selected from the group comprising of pharmaceutically acceptable glycols selected from propylene glycol, hexylene glycol, diethylene glycol monoethyl ether, alcohols, polysorbate 40, 1, 2-, diols, and suitable mixtures thereof.

15. The topical composition as claimed in claim 10, wherein preservative is selected from acids, esters, alcohols, phenols selected from butylated hydroxy anisol, butylated hydroxy toluene, and quaternary ammonium compounds.

16. The topical composition as claimed in claim 10, wherein propellant or surfactant is selected from the group comprising of hydrocarbons selected from propane and butane, a chlorofluorocarbon dimethyl ether, hydroflourocarbon and suitable mixtures thereof.
